(19) United States
(12) Patent Application Publication
(10) Pub. No.: US 2003/0113727 A1
(43) Pub. Date: Jun. 19, 2003

(54) Family history based genetic screening method and apparatus

(76) Inventors: Kanwaljit Singh Girn, Heston (GB); Craig D. Livingstone, Benson (GB)

Correspondence Address: RATNERPRESTIA, P O BOX 980, VALLEY FORGE, PA, 19482-0980, US

(21) Appl. No.: 10/010,706

(22) Filed: Dec. 4, 2001

Related U.S. Application Data
(60) Provisional application No. 60/251,532, filed on Dec. 6, 2000.

(51) Int. Cl. C12Q 1/68; G06F 17/60; G06F 19/00; G01N 33/48; G01N 33/50

(52) U.S. Cl. 435/6; 702/20; 705/3

(57) ABSTRACT

A computerized process by which an individual may generate family history information for use by a healthcare provider in determining whether the individual is a candidate for genetic testing prompts an individual to enter family history information and receive a computer-generated evaluation of that information. If the evaluation indicates that the individual is at risk of developing a genetically-linked illness, the process prompts the individual to schedule genetic counseling. Pending the result of the genetic counseling, genetic testing is scheduled for the individual.

### CROSS-REFERENCE TO RELATED APPLICATIONS

This application claims the benefit of U.S. Provisional Application No. 60/251,532, filed Dec. 6, 2000, the contents of which are incorporated herein by reference.

### BACKGROUND OF THE INVENTION

The present invention concerns genetic screening and, in particular, an on-line family history, health status and lifestyle screening system that may be used by individuals or health-care professionals to reduce unnecessary health-care expenditure and, where certain risks are identified, help avoid the incidence of, or enable early intervention in the treatment of genetic disease.

Advances in the field of Human Genetics will revolutionize medicine in the 21^{st} Century. Already many thousands of hereditary diseases have been identified. More than 10,000 diseases are currently listed in the Online Mendelian Inheritance in Man (OMIM) database. Output from the recently published Human Genome project draft will enhance our knowledge of the hereditary factors that contribute to disease and disease risk. The coming years will see the development of new tests and clinical interventions as more and more health-associated mutations are identified.

According to Cyberdialogue, 23 million individuals in the US searched for healthcare information on the internet in 1999 and this was forecast to rise to 30 million in 2000. Fifty-two percent of individuals retrieving healthcare information on-line are searching for disease information.

Many high prevalence (and high economic impact) diseases have a known or suspected genetic component. One such disease that is suspected of having a genetic component is asthma. In the United States alone, 10 to 15 million people have a predisposition toward this disease.

One method for determining if an individual is genetically predisposed to develop a given disease is to perform a genetic test for a mutation that has been associated with that disease. It is estimated that the demand for DNA testing could rise from $200 million to $1.4 billion by 2003 due to falling prices for DNA tests. This will have a significant impact on the economics of healthcare systems and will impact their ability to deliver care.

The pace of technological change in the development of new genetic tests is not being met by a similar increase in the number and capacity of genetics professionals able to help individual healthcare service users to identify, understand and manage their genetic risk, “genetic counselors” in the USA. Currently, There are only about 1,000 board certified genetic counselors in the US.

A report produced in 1997 by the US Task Force on Genetic Testing noted:

“The rate of increase of health care professionals trained and board-certified in medical genetics or genetic counseling has not kept pace with the rate of increase of genetic discovery and of potential demand for genetic tests . . . If the demand for genetic testing increases, and the supply of genetics providers does not keep pace, other health care professionals will have to play a role, or new models of testing will have to be devised if the demands are to be met.”

Yet currently only a small proportion of Healthcare Organizations provide genetic counseling services. For those that do provide these services, capacity is likely to be reached or exceeded in the near future.

Given the public awareness of diseases with genetic links and the availability of genetic testing, it is unlikely that Healthcare organizations will simply refuse to cover the tests. Given the expense of the tests, however, it is likely that these organizations will want to ensure that they are administered only to those who would truly benefit from the tests. One preliminary step, therefore maybe to ensure that their members get professional counseling prior to testing.

The demand for genetic testing, however, could be very large. The inventors have determined that many tens of millions of Americans have a close family member who suffers from one or more of the diseases it intends to cover. Given the relative scarcity of trained genetic counselors, and the current lack of training for more generalist healthcare providers, it is difficult to determine how this need will be met. One option may be to place the burden on primary care professionals. Because they are often the closest contact point with their patients, family physicians already discuss the inherited or familial component of conditions with at least a few patients, yet many of these physicians have no formal training in genetics.

Consequently, there is a tendency among primary care physicians to refer patients to secondary care even though studies have shown that:

“70% of individuals are able to accurately record their family history”

“Accurate family histories reduce physician consultation time by around 48%”

“Pre consultation collection & analysis of familial data reduces referral into secondary care by 46%”

“A patient who is incorrectly advised to see a genetic counselor takes four to seven hours of the counselors' time at a cost of $150 to $200 per hour.”

One factor that may impede an individual from taking a genetic test is the fear of genetic discrimination. The individual may fear, for example, that the healthcare provider may revoke health insurance if it discovers a genetic predisposition to a particular illness. The individual may also fear loss of employment if the employer finds out the results of the test. Accordingly, any genetic testing desirably offers security and confidentiality. Healthcare providers should agree to keep the results confidential and not to act on the results in a way that could prejudice the individual's or their family's ability to obtain insurance and other services. This is likely to be a prerequisite for an individual participating in any procedure other than basic genetic education, e.g. risk assessment, testing or genetics based clinical interventions. To further illustrate the desirability of procedures including genetic testing, a few specific diseases are described below:

The genetic link with breast cancer was discovered in 1994 when the breast cancer susceptibility gene, BRCA1, was identified. A second, entirely different breast cancer susceptibility gene, BRCA2, has also now been discovered. Each year about 175,000 cases of breast cancer occur in the US. Even though the American Cancer Association recommends yearly mammography and monthly self-examination for women aged 40 and above, less than 50% of women over 65 have ever undergone mammography, and an even smaller proportion is screened regularly.

It is estimated that if diagnosed and treated early, 90% of breast cancers are curable. The treatment for early breast cancer costs $26,500-33,000, whereas for late breast cancer, the cost is $92,500-$106,000, with chemotherapy for 6 months adding a further $4,400-6,600. The only clinically available comprehensive BRCA1 & BRCA2 test available is BRACAnalysis which costs around $2,500.

Ovarian cancer is the seventh most common cancer in women. It ranks fifth as the cause of cancer death in women and is associated with high mortality because it is often discovered at an advanced stage. The American Cancer Society predicts that there will have been about 23,100 new cases of ovarian cancer in this country in the year 2000.

Of these, about 14,000 women will die of the disease. The chances of survival from ovarian cancer are better if the cancer is found early. If the cancer is found and treated before it has spread outside the ovary, it is estimated that 95% of women will survive at least five years.

Using existing procedures, only 25% of ovarian cancers, however, are found at this early stage. The American Cancer Association recommends that all women should have the following examinations: women aged 18-40 should have a pelvic exam by their doctor every 1-3 years, Women over 40 should have a yearly check-up with a pelvic exam. Unfortunately, compliance with this recommended regime is about on par with that for breast cancer. Widespread genetic testing or genetic counseling may be able to identify those who would most benefit from the examinations, allowing healthcare providers to intensify their efforts with regard to those specific individuals.

Colorectal cancer is the second most common form of cancer in the U.S. It has the second highest mortality rate, accounting for about 140,000 new cases and about 55,000 deaths each year. An individual's lifetime risk of dying of colorectal cancer in the U.S. has been estimated to be 2.6%. A survey in 1998 by Gallup for the National Colorectal Round Table revealed that nearly half of all adults aged 50 and older—the age group considered at highest risk for developing colorectal cancer—have not been screened for the disease. Fear and embarrassment are one factor, but so too is the failure of physicians to recommend screening. Again, if individuals who could be identified as having an increased genetic risk for developing colorectal cancer could be identified, healthcare organizations could better target their early detection resources.

High Cholesterol is a metabolic disorder. This is the result of the overproduction and/or under-utilization of Low-Density Lipoprotein (LDL). It is a common disorder: it is seen in 1 in 500 individuals. High Cholesterol can also be caused by the consumption of a high-cholesterol diet. The human body usually produces about two-thirds of its needed cholesterol in the liver, thus very little supplement of cholesterol is required. It is generally believed that the typical high fat, high cholesterol, high protein, low fiber U.S. diet is largely responsible for 600,000 deaths annually. Obesity and a sedentary life style add to the risk. If individuals at risk for developing high cholesterol could be identified, targeted interventions which encourage a more healthy lifestyle may both increase individual's quality of life and reduce long-term costs for the healthcare provider.

The diseases discussed above are only a small selection from the thousands of diseases which have been identified as having a genetic component for which knowledge of the risk of developing the disease would be beneficial both to the individual and to the healthcare provider. They demonstrate, the need for a method of identifying individuals who are at risk of developing such a disease that would be used by the individuals.

Individuals are embracing telemedicine, which allows them to take more responsibility for their health and healthcare decisions. According to Cyberdialogue's Cybercitizen Health Survey in October 1999: 48% of patients want to communicate with their doctors by e-mail, and a third of those who want to communicate in this way would switch doctors to get these services. This embracing of telemedical communication with the medical profession by patients as well as within the profession offers a means to relieve the economic impact and potential bottlenecks brought about as a result of the increased ability to detect and intervene in disease (and disease risk).

### SUMMARY OF THE INVENTION

The present invention is embodied in a computerized process by which an individual may generate family history information for use by that individual, who may work with or without a genetic counselor, or by a healthcare provider in determining whether the individual is a candidate for specific screening methodologies, including genetic testing, or specific medical interventions, such as prophylactic mastectomy.

The present invention may be presented as a single “stand-alone” or other local use computer system or as a client/server or internet based or other similar remote access computer system.

According to one aspect of the invention, the individual selects to record information relating to a particular disease or group of diseases (e.g. a specific disease may be “breast cancer” or a group of diseases may be “breast cancer, ovarian cancer, pancreatic cancer and endometrial cancer.” Another disease group may be “cardiovascular disease” which may cover “heart attack, stroke, inherited lipid disorders etc.”) in which a family history (“Pedigree”) with current health status data will allow individual risk of developing that disease to be determined.

A process running on the computer (either the computer they are using or a remote “server” computer) guides the individual to collect data comprising his/her pedigree (family tree). For each member of the family in this family tree, the individual is asked to provide demographic together with historical and current health status data that is specific to the selected disease. The process then analyzes the information provided in the context of the pedigree to determine if the individual is at risk of developing the selected illness.

According to one aspect of the invention, the process may include an appointment scheduling facility that allows the individual to make an appointment with a genetic counselor after receiving the result of the determination. That appointment may be “face to face” or may use telemedicine techniques, including the interactive sharing of access to the system embodying a process according to the present invention.

According to another aspect of the invention, the computer system includes informational materials concerning the selected illness and these informational materials are selected and presented to the individual on the basis of the data collected or the analyses performed. The information may offer education or guidance or support a decision as to whether a particular diagnostic or other medical intervention may be desirable (e.g. genetic testing) or if a lifestyle change may be beneficial in reducing estimated risk.

According to yet another aspect of the invention, information concerning the identity of the individual may be maintained between sessions on a local computer system (the “client”) and need not be wholly transmitted to a remote computer system (the “server”) for persistent storage with the aim of protecting the anonymity of the individual.

According to a further aspect of the invention, the system may contain an access control mechanism allowing roles based access to clinical data, allowing appropriate access by patients, clinical genetics specialists, non-genetics-expert clinicians and systems management personnel.

According to a further aspect of the invention, the system may contain an access control mechanism that allows sets of clinicians to manage more than one patient's (or other healthcare system user's) family history and that allows each patient (or other healthcare system user) to store and manage more than one family history (for example covering different diseases or different versions of unknown information concerning a single disease).

### BRIEF DESCRIPTION OF DRAWINGS

FIG. 1 is a block diagram of an exemplary system architecture suitable for use with the present invention.

FIG. 2 is a flow-chart diagram which is useful for describing the overall operation of an exemplary embodiment of the present invention.

FIG. 3 is a flow-chart diagram of the data entry step shown in FIG. 2.

FIG. 4 is a flow-chart diagram of the data entry step of FIG. 3.

FIG. 4A is a flow-chart diagram of the display and edit step of the process shown in FIG. 4.

FIG. 5 is a flow-chart diagram of the data edit step shown in FIGS. 3 and 4.

FIGS. 6A, 6B, 7A, 7B, 7C and 7D are screen prints generated from the exemplary system that are useful for describing the data entry step shown in FIGS. 2, 3 and 4;

FIGS. 8A, 8B, 9A and 9B are screen prints generated from the exemplary system that are useful for describing the display and edit step shown in FIGS. 4, 4A and 5; and

FIGS. 10 and 11 are screen prints that are useful for describing the perform analysis and generate reports steps shown in FIG. 2.

FIG. 12 is a screen print of an extract from a risk report.

### DETAILED DESCRIPTION

The present invention is embodied in a system that provides easily accessible healthcare guidance, screening, triage and risk assessment services aimed at members of the general public, their non-genetics specialist and genetics specialist healthcare providers. This service may be extended to include, monitoring compliance with preventative regimes, and mediation of discussions of the interventions and lifestyle options with care givers either locally or remotely.

A service according to the present invention is designed to provide genetic counseling and basic clinical genetics services more cheaply to a wider population. It also reduces the treatment cost of chronic care through earlier detection, reduces the number & costs of medically unnecessary diagnostic testing through more frequent screening, and enhances member benefits through the provision of comprehensive genetic services.

The present invention combined with genetic testing, when recommended, offers a method for identifying the risk of an individual of developing conditions that have a genetic component. It is a means of anticipating later treatment needs now, thereby enabling action to be taken that will both cut the cost of treatment and improve patient outcomes. The present invention offers a “predict and provide” approach to genetic testing at two levels, it allows individuals and their physicians to identify risk and take avoiding action and it allows healthcare organizations to recognize and manage future costs on a population basis.

The inventive service is based primarily on population studies of inheritance patterns for common late-onset genetic diseases. It is likely that the service will be enhanced both in the number and types of diseases it covers and in the methods for identifying a genetic predisposition to a particular disease as a result of the identification of the molecular genetic causes of these diseases coming from the Human Genome Project and related research efforts. The service is applicable to “simple” genetic disorders as well as complex, late onset disorders.

This section outlines the science underlying the present invention, covering basic genetics and the importance of family history. The most fundamental diagnostic tool in clinical genetics is the pedigree (family history). Clinical geneticists regard it as the gateway to recognizing inherited disorders. All of the many thousands of hereditary conditions are susceptible to the family history approach. In addition to the diseases listed above, these include such common “complex genetic” conditions as cancer, diabetes, heart disease, Alzheimer's disease and mental illness, as well as genetic disorders of a “simple genetic” character such as Hemachromatosis, Thalasemias, Tay Sachs disease etc.

The present invention presents the family history graphically as a pedigree (family tree) using standard symbols. Health professionals working in family practice, internal medicine, endocrinology, pediatrics, and obstetrics have found this visual pedigree (family history) representation and the associated screening tools very useful for identifying patterns of inheritance, calculating risks, distinguishing genetic risk factors from other risk factors, deciding on testing strategies and making a diagnosis of a specific disease. The family history is also useful to determine reproductive options, make decisions on medical management and surveillance, developing patient rapport and generally educating the patient.

An important aspect of the inventive risk assessment service is the automation of the process of collecting family histories. Reviewing a family history can aid the clinician in diagnosis. For example, in making a diagnosis of a familial cancer it is important to know, how different types of cancer are grouped in a family history, the ages of the individuals diagnosed with cancer and how closely the individuals with cancer are related to each other (i.e., close members of the family as compared to more distant relatives).

The ability to obtain access to information about the disease and to schedule an appointment (either by telephone supported by the inventive system described here or in person, “face to face,” again supported by our inventive system) with a counselor soon after seeing the results of the genetic risk screen is one aspect of the invention. This aspect of the invention can provide emotional support and guidance to the users of the invention, allowing them to make the appropriate choices to meet their own unique needs.

The online counseling service enables consumers to request telephone-based counseling, via the on-line service, on the implications of their risk profile and how to mitigate that risk. The service embraces the trend towards telemedicine, reducing the need for the consumer to travel to the counselor. Consumers with a need for more formal counseling, however, may be referred to appropriate referral centers offered either by their healthcare organization or through the inventive risk assessment service.

The inventive risk assessment service enables a user to assess the risk of developing a genetic disease based on family history and other factors. The exemplary embodiment described below includes breast cancer, colon cancer, ovarian cancer and heart disease. It is contemplated, however, that the system may be extended to several other high prevalence diseases such as diabetes and Alzheimer's disease.

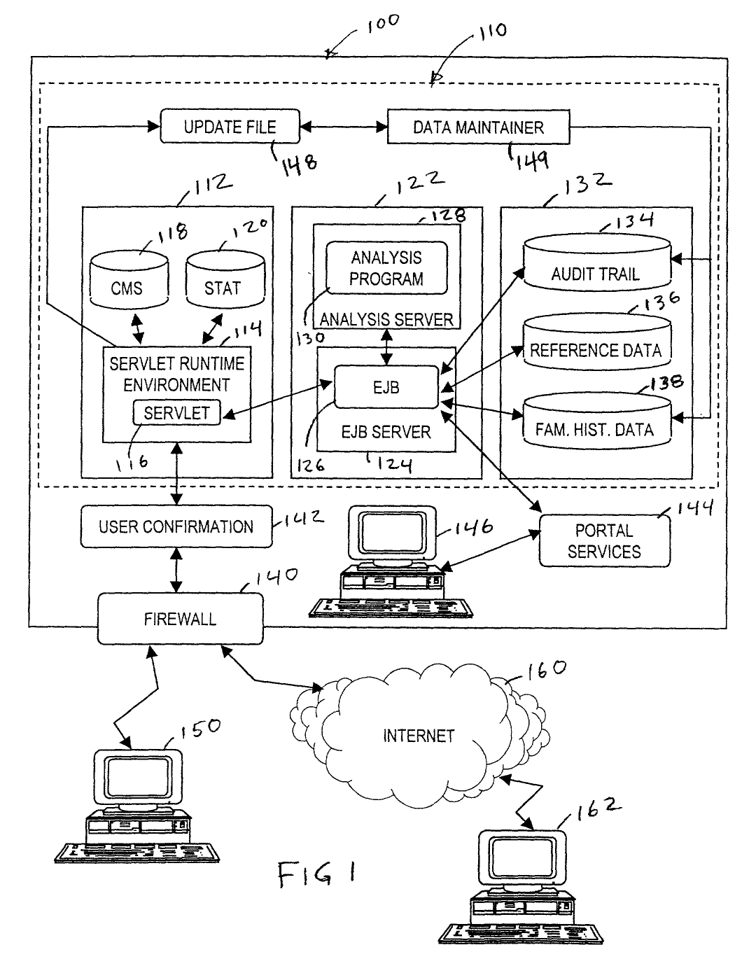

FIG. 1 is a block diagram that shows an exemplary hardware and software system suitable for use with the present invention. As described above, the present invention may be implemented in a variety of environments: as a single machine providing a local service, as a subdomain of a healthcare web site, providing service to a local area, as an adjunct to a healthcare provider's web site, serving its patient and provider population from computers within the facility, from on-site kiosks or from the users' homes, as a service provided to members of the public by their health plan in their homes or at their physicians offices, and as an independent “consumer genomics” portal etc. It is desirable, however, that the invention have a single architecture that can be readily adapted to each of these environments.

In addition to supporting the variety of environments, it is desirable for the system to be scalable for use by relatively small healthcare providers, such as a healthcare service operated by several physicians from a single office, to much larger providers, such as a multi-state or national health maintenance organization (HMO). Ideally, the system should be independent of the hardware and operating system used by the healthcare provider and should also be readily integrated with the provider's legacy systems. The inventors have determined that an exemplary large system, supported from a single “low end” server should support as many as 65 concurrent users (i.e. 65 simultaneous actions).

To meet these various constraints, the present invention is implemented in three layers: a presentation layer 112, an applications layer 122 and a data layer 132. These layers roughly correspond to the open systems interconnect (OSI) presentation and applications layers with the data layer being an additional layer above the applications layer. All of these layers may be implemented on a single computer (for a low-use system) or each layer may be implemented using multiple servers (for high-use systems). The modularity of the exemplary system architecture allows the system to be easily scaled to meet the particular requirements of a large variety of possible implementations.

For maximum portability, the presentation layer 112 is implemented using servlets. Servlets are the web server's equivalent to applets for a user's browser. One difference between servlets and applets, however, is that servlets do not have any graphical user interface (GUI) components. Servlets are written using functions provided by a servlet applications program interface (API). This API is provided in the web server for the portal server 100. Because servlet API's are available for many commercial web servers, the family history and counseling software according to the subject invention may be implemented at a particular healthcare provider's site with minimal programming. Additionally, it can be implemented using the favorite web server, enterprise java beans middleware and database engine in use at that site, again with minimal modification.

In the system shown in FIG. 1, the present invention is implemented as a subdomain 110 of a “healthcare portal” web site 100. In addition to the family history and genetic counseling functions provided by the subject invention, the healthcare portal site may provide other services to members of the healthcare provider. These may include, for example, lists of primary physicians and specialists who are in the service, information on diet and exercise programs and health-related news items. Consumer confidence in the family history product is enhanced if it has the same “look and feel” as the portal site. Accordingly, display screens for the subject invention are desirably implemented using the toolkit for the portal site. The flexible authoring and publication approach described above allows this “look and feel” to be maintained in implementing the inventive service.

Because a portal site 100 has its own security measures to ensure that only valid users have access to data on the site, the subject invention assumes that all users are valid users. That is to say, the present invention relies on the portal site to confirm that a particular user is entitled to access the family history database. Consequently, the subject invention does not receive or maintain any identifying information about the users. Each user is identified by a unique identification number. The identification number may be provided by the portal site, if the family history system is used by healthcare providers within the healthcare domain, or it may be generated and stored locally on the user's computer with other identifying information, for example, as an Internet “cookie.” In this instance, software external to the subdomain 110 handles the creation of the cookie file, although a servlet 116 may be programmed to extract only the identification number from the cookie file.

As shown in FIG. 1, the family history and genetic counseling functions of the present invention are implemented in the subdomain 110 which is inside of the firewall 140 of the healthcare portal 100. Also as shown in FIG. 1, external users may gain access to the subdomain 110 either by a direct connection between the user's computer 150 and the firewall 140 or by connection from a remote computer 162 via a global information network 160. such as the Internet. Once inside the firewall, the healthcare portal host performs any necessary checks to confirm the validity of the users and whether they are allowed access to the subdomain 110.

Although the user's computer and the remote computer 162 are shown as separate devices, it is contemplated that either of these computers may be implemented as a separate computing environment in the computing system of the health care portal 100, but outside of the subdomain 110, for example in an application services provider (ASP) environment. The system may also be implemented on a single computer system for “desktop use.”

Other implementations may make use of a flexible but robust security model as described in more detail below. Access may be controlled by username/password authentication. The software, in another exemplar implementation, offers roles based access control. The level and range of access to data is dependent on the role of the individual whose username and password are used to access the system.

The software separates clinical data from patient identifying data, preventing even staff who maintain the system from easily compromising patient privacy without appropriate access. Clinical and/or patient identifying data may also be encrypted while stored in the system. The encryption key may be held by a responsible third party (e.g. the medical director at the institution using the software). Staff attempting to compromise the encryption, for example manual application by IT staff of the decryption key to data held in the clinical and patient database, would be subject to disciplinary procedures.

The software can, additionally, be placed within the context of an existing security framework used to control access to clinical applications at a site.

For optimum security, all information received from and provided to a user is, desirably, strongly encrypted during transmission. This may be implemented, for example, through the use of hypertext transmission protocol, secure (HTTPS) protocol or other encryption protocol implemented, for example, in a secure sockets layer (SSL) environment.

The system implements an audit trail. This can show the time at which the system was accessed, by whom and what data they added/deleted/changed. Implementations of the system allow roll-back of data items to previous settings so that changes can be more fully tracked.

Not all of these levels of security are implemented in every deployment of the software, but the software is capable of meeting current security requirements and the provisions recommended in, for example, the UK's Caldecott report or under the HIPPA regulations in the USA.

Because the exemplary family history system does not need to maintain any identifying information about the users, the data it obtains can, by choice of the using institution or the individual user, be completely anonymous. An anonymised data extract may be collected and provided to the hosting institution for analysis to determine its future needs without compromising the identity of any of the users. In addition, the data may be processed to further refine family-history-based heuristic measures that are used to define the risk of contracting a genetically linked disease.

If the user is allowed access to the family history and genetic counseling services of the subject invention, a connection is established by a servlet 116 of the servlet runtime environment 114. The servlets in the presentation layer 112 receive user data and control the user interface through JavaScript routines provided to the user's browser. In addition, the servlets provide the user with access to content such as educational materials on the various diseases that is maintained in the content management system (CMS) database 118.

Each item of information presented to the user is available translated into several languages for easy use both by subscribers who access the system from other countries and for minorities within the home country of the healthcare provider. The exemplary content management system 118 contains and maintains this content as well as the text of the on-screen commands in each of the supported languages. The multi-language nature of the system can extend to the presentation of each item of the user interface. For each item, several versions may be selected from, each supporting one of the supported languages.

All encoding and decoding performed by the system is implemented through the servlets 116 of the servlet domain 114.

In addition to the CMS 118 The servlet domain 114 also maintains a statistics database 120. The exemplary statistics database stores details on the usage of the inventive family history and counseling system to allow analysis of the performance and availability of the system. The data stored also includes time metrics for user interaction with the system. Using this information, software developers can continually improve performance of the system and readily determine the likely effect of any new features. It is contemplated that the statistics database 120 may be implemented as a flat file log which is analyzed and reset on a weekly basis as an administrative task.

The servlets in the servlet runtime environment also maintain an update file 148 which records any and all changes to the data entered into the system and maintains a record of all accesses to the data. This update file is periodically examined by a data maintainer 149 which records relevant information into an audit trail database 134 in the database layer 132. The data maintainer also extracts compressed versions of any family history data that is entered from the family history database 138 and stores this data in the audit trail database 134. The audit trail database allows system administrators to determine the guidance given to a particular user based on the recorded information that was entered by that user. The contents of the audit record for a particular user are described in more detail below.

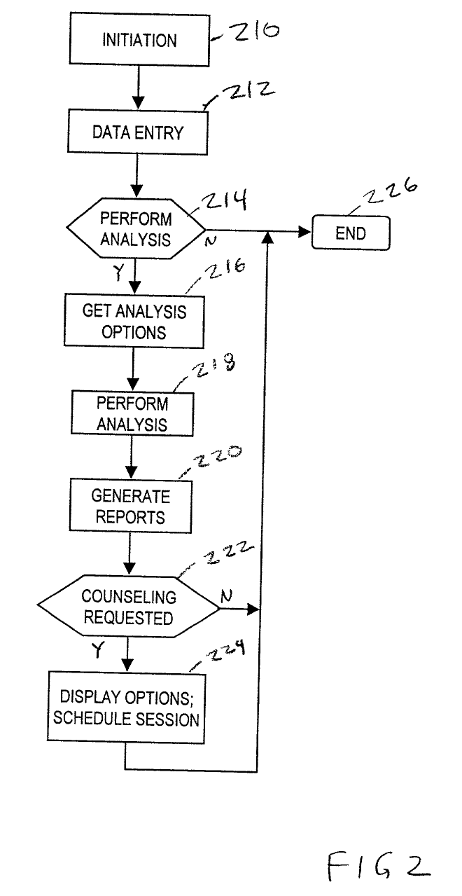

After the user has entered the data for his or her family history, as described below with reference to FIGS. 2, 3, 4 and 5, the servlets 116 format the data and pass it to the applications layer 122. The exemplary applications layer is implemented, for example, using Enterprise Java Beans (EJB). The Enterprise Java Beans are reusable software units that are written using a Java Beans API. The methods used by Java Beans are no different from Java methods. The applications layer 122 combines the Java Beans into servlets that communicate with the servlet runtime environment 114. The Beans may directly perform the processing functions described below with reference to FIGS. 2 through 5 on the raw data obtained from the presentation layer 112 or they may be combined into servlets which perform these functions. Enterprise Java Beans also allow for interprocess communication so that multiple parallel processes may be launched as separate threads that communicate with each other through events. This facility allows the applications layer 122 to be scaled to handle multiple users, for example, by providing multiple presentation layer servers which communicate with multiple analysis servers through the EJB environment.

The applications layer receives the decoded data from the presentation layer, forms the family history, and passes the family history on to the analysis server.

The analysis server, in the exemplar implementation, shows how the inventive system can integrate with several risk analysis programs, including: a family history statistical risk analysis program written in Fortran; and a statistical risk analysis program focussed on breast cancer. In addition, the analysis server includes heuristic algorithms that are used to identify possible genetic risks associated with other genetically linked diseases. When the user selects a particular disease a risk analysis program is also selected. After the user has entered the family history data, the EJB server 124 invokes the appropriate analysis program from the analysis server and retrieves the results. The results are then sent back through the servlet runtime environment 116 to be encrypted and transmitted to the user for display. The family history and the results of the analysis are also stored in the family history database 138 and a compressed family history, an indication of the analysis program that was invoked and a compressed version of the results are also stored in the audit trail database 134. The reference database 136 holds all accessory data that is used by the analysis programs. This database has a flexible structure to meet the data requirements of each of the analysis programs.

A final link from the EJB server 124 is to the portal services 144. This link allows users 146 inside the firewall 140 as well as administrators access to the family history and counseling system. In addition, the exemplary portal services 144 is implemented as a Common Object Request Broker Architecture (CORBA) wrapper to allow users 150 and 162 outside of the firewall 140 access to services offered on the portal system through the inventive family history and counseling system.

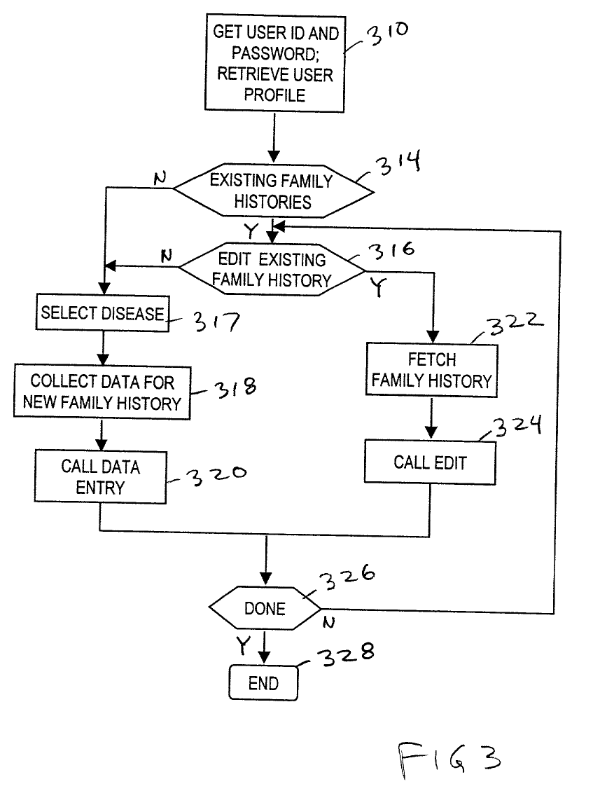

The processing environment shown in FIG. 1 is used to implement the exemplary family history and counseling system. FIG. 2 is a flow-chart diagram of the overall operation of the exemplary embodiment of the invention. The process begins at step 210 when the user initiates data entry at step 212. As described below with reference to FIGS. 3, 4 and 5, the user enters a particular genetically linked illness and edits his/her family history data in the data entry step 212. At step 214, once the data is entered, the system determines if the user wants to perform a genetic analysis on the entered family history. If so, step 216 determines the type of analysis that is appropriate for the specified genetic illness and accesses the data for that analysis from the reference database 136. At step 218, the system invokes the analysis program, passing the user's family history and the data retrieved from the reference database 136. The analysis server 128 returns the results of the analysis at step 220 and the system formats the results into a report. When the system presents the report to the user, it also allows the user, at step 222, to request an appointment with a counselor. This appointment may be for an on-line consultation, a telephone consultation or an office visit. The user selects the desired type of counseling and schedules the session at step 224. After step 224 or, if at step 214, the user does not want to perform an analysis of the family history or, at step 222, does not request counseling, the process ends at step 226.

FIG. 3 is a flow-chart diagram of the initiation step 210 and data entry step 212 of FIG. 2. The first step, 310, gets and verifies the user's identifier and password and verifies that the user is allowed access to the system. Also at step 310, the system uses the identifier to retrieve the user's profile from the family history database 138.

After step 310, the process executes step 314 which determines whether the user wants to edit an existing family history or enter data for a new family history. If the user wants to enter a new family history, then at step 317, the system allows the user to select a particular disease for analysis. Each genetically linked disease uses a different family history because questions asked about each individual in the family history differ for each disease. Next, at step 318, the process collects the data for the new family history from the reference database. This data includes questions to be asked and the particular analysis process to be used and the parameters for that process. After and during the data collection operation, the user is prompted to review and edit the data at step 320 to ensure that it is correct.

If, at step 316 the user chooses to edit an existing family history then, at step 322, the user selects a particular family history from the family history database 138 and, at step 324, edits the family history. At step 326, the process determines whether the user wants to enter or edit another family history. If so, control transfers to step 316, described above. Otherwise, the data entry operation ends at step 328.

The collection of information leading to the construction of a “pedigree” or “family tree” of the relatives of an individual for whom a risk assessment may be performed by either of two methods, described below.

The first is interrogative. The interrogative route starts with the individual who is using the computer system (“You”). You are asked to record the number of siblings you have and their sexes. You are then asked the number of siblings each of your parents had and their ages. The system can then ask about the number of children of each sex, you, your brothers, your aunts and uncles etc. have, about your grandparents and their siblings and the offspring of their generation and so on. This iterative process guides you through your entire family, the answers to each question defining the next set of questions to be asked. Using this method, an entire family tree can be defined including all individuals who are within a certain number of degrees of relatedness of “you”. (The degree of relatedness is determined by the number of “meioses” or combinations of parental genetic material different an individual in the family is from you. Your “first degree relatives,” for example are one meiosis different from you—your full brothers and sisters, your biological children and your birth parents.) The number of degrees of relatedness for which data is gathered is determined by the size and quality of the data from population studies that support risk assessment for the target disease or by the requirements of the guidelines being used to assess risk. The interrogative method accounts for inbreeding within families (intergenerational and other marriages between related individuals), twinships and multiple births and for re-marriage and resulting sets of children that share only a single parent

The second method is user driven. The user is presented with a picture of their family tree or pedigree that summarizes all of the data collected at a given stage in the process. The user may choose, for example, to add a brother, sister or parents to an individual, and may, by interacting with a set of drawing tools describe their entire family structure, again accounting for inbreeding within families (intergenerational and other marriages between related individuals), twinships and multiple births and for re-marriage and resulting sets of children that share only a single parent. This visual, user driven method may be used alone or in conjunction with the interrogative method.

While maintaining confidentiality, the service may request the first names of family members, as a way of allowing easy identification of individuals referred to in subsequent questions. For example, if the name of an individual is John, the system asks about John's father, mother, eldest sister, etc. If the system knows that John's father is Eric, it can then ask about Eric's mother, daughter, son etc. by reference to Eric.

For cancers, the system gathers information on occurrences and reoccurrences of breast, bowel and ovarian cancer, uterine and endometrial cancers, benign bowel cancers and other childhood cancers. For high cholesterol, details of a first myocardial infarct, cholesterol levels and physical signs are gathered for each individual in the family history.

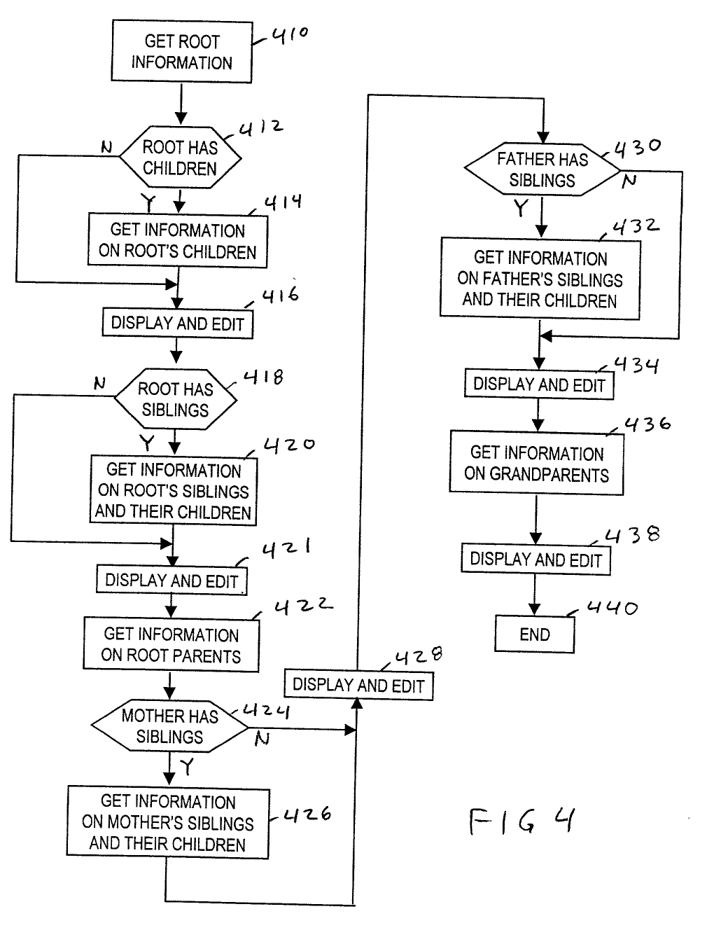

FIG. 4 is a flow-chart diagram which is useful for describing the data entry step of the process shown in FIG. 3. The data entry process is used to gather data about a new family history. It begins at step 410 by obtaining information about the “root” individual, that is to say, the individual whose risk for developing a genetically linked disease is to be determined, also known as the “index case” or the “proband”.

The collection of data relating to each person in a pedigree may comprise, but is not limited to (a) demographic data, such as name (although this is not strictly required and may not be used in order to secure anonymity, as described above), date of birth, mortality status (alive/dead), their ethnicity, their membership of a multiple birth (twinships, triplets etc) (b) medical history data. This information is gathered because some diseases are more common in some ethnic groups than in others.

For the recording of a medical history we use cardiovascular disease (heart disease and stroke) here as an example. In cardiovascular disease, medical history data would include but not be limited to: whether they had had a heart attack, at what age, whether they had had more than one heart attack; Whether and at what age they had had a stroke, and if they had had more than one episode; Whether certain medical interventions or tests had been carried out on them (and if so whether it had been performed more than once) and their age at the time (e.g. angioplasty, heart bypass, heart transplant, carotid ultrasound, heart scan, treadmill test, thallium scan). Abnormal test results are captured. Diabetic status is investigated, as is blood pressure and thyroid status. Current and prior medication data is also captured, especially with regard to lipid lowering medication. Physical signs and symptoms associated with abnormal lipid metabolism are recorded (e.g. overweight, xanthelasma, xanthoma, arcus senilis etc.). Data on lifestyle may also be recorded, e.g. smoking and exercise. For women in the pedigree, their menstrual status and/or use of HRT may be investigated and recorded. All of these questions are asked in a “contingent” manner. For example, with regard to heart attack, the system may ask, “Have you ever had a heart attack.” Depending on the answer, for example, Yes/No/Don't Know, the system may seek more information, e.g. “At what age” or “Have you had more than one heart attack?” following a pattern of questioning that matches how a doctor would investigate a medical history. The amount of detail sought for each individual in the pedigree may be varied, for example by degree of relatedness, to match what “you” are likely to know about a relative of a certain degree of kinship.

It is important to note that the results of previous genetic testing can be included in the family medical history and can be accounted for in the analysis performed, as described below.

Questions about individuals may be asked (i) as the pedigree is built up in the “interrogative” version of the pedigree builder (described above), (ii) as each individual is added in the “user driven” version or (iii) as a separate stage, in a second pass through the pedigree

The present invention comprises a system that can support the creation of any family history or pedigree structure and can collect data relating to a disease or disease group. The process and supporting system do not vary. The specific data to be collected is set in a specific configuration of the supporting system. This process may, therefore be applied to all other diseases in which there is a component of risk of a familial or genetic nature. In the example implementation of the system, this flexibility is gained by the use of Dynamic HTML and XML techniques in the configuration and presentation of questions, and in the storage, manipulation and presentation of the resulting data.

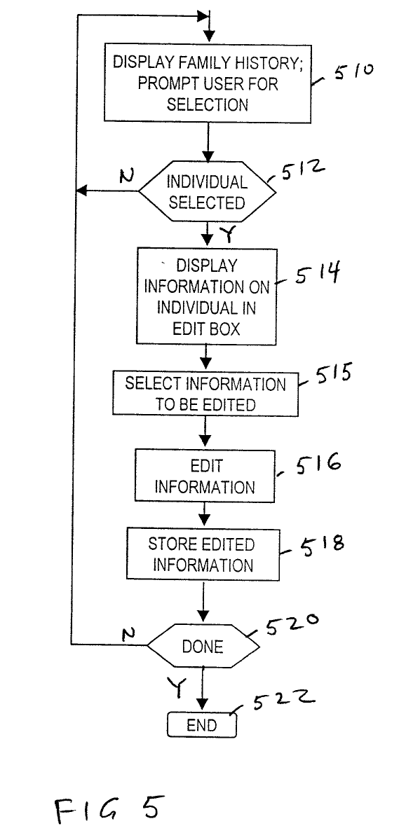

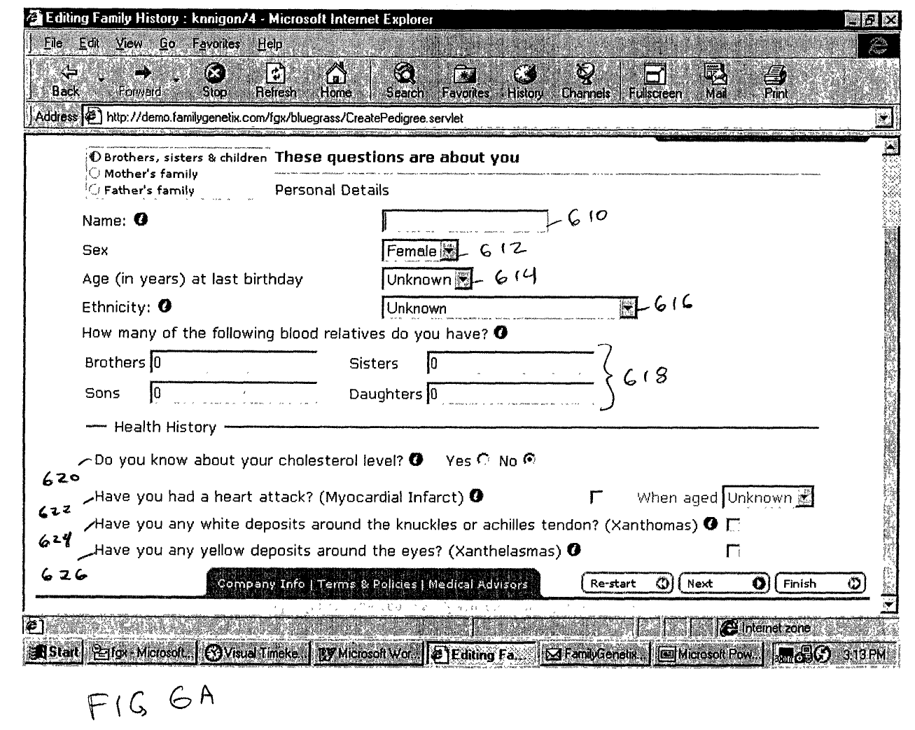

A screen-print of an exemplary web page used to gather data about the root individual is shown in FIG. 6A. This web page is generated by a servlet 116 from the servlet runtime environment 114 of the system shown in FIG. 1. The page includes fields for entering the first name of the individual, 610; the sex, 612; age, 614; and ethnicity 616 of the individual as well as a set of fields 618 for enumerating the individuals brothers, sisters, sons and daughters. The page also asks questions specific to the selected genetic disease, in this example, familial hypercholesterolemia (FH). These questions ask the individual about his or her cholesterol level 620, whether he/she has had a heart attack 622, whether he/she has any white deposits around the knuckles or achilles tendon 624, and whether the individual has any yellow deposits around the eyes. FIG. 6B is a more complex exemplary implementation of the web page used to gather data about the root individual. This form may be used, for example, by a clinician. It is contemplated that in more complex implementations, a clinician may be able to gather more detailed information about each member of the pedigree. Data may also be gathered from administration, clinical and laboratory systems to provide a more complete picture of the risk for a family. Clinicians may be offered the more complex form, shown in FIG. 6B, while patients are offered the less complex form, shown in FIG. 6A. The exemplary system may also be configured to allow different patterns of data sharing between clinicians and between clinicians and patients. This sharing is desirably governed by both a hierarchy of access and access based on the role of the individual (e.g. as a patient or a clinician).

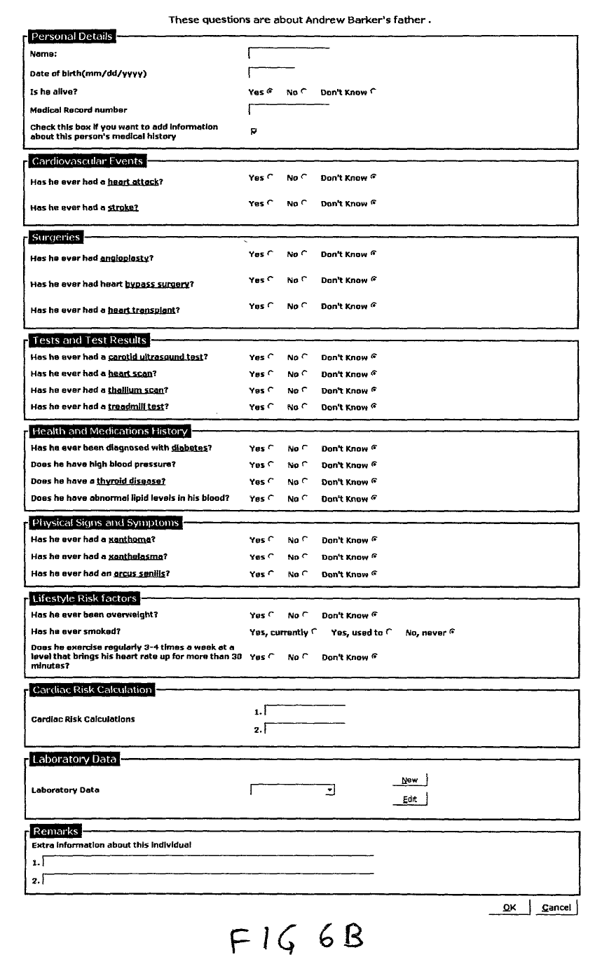

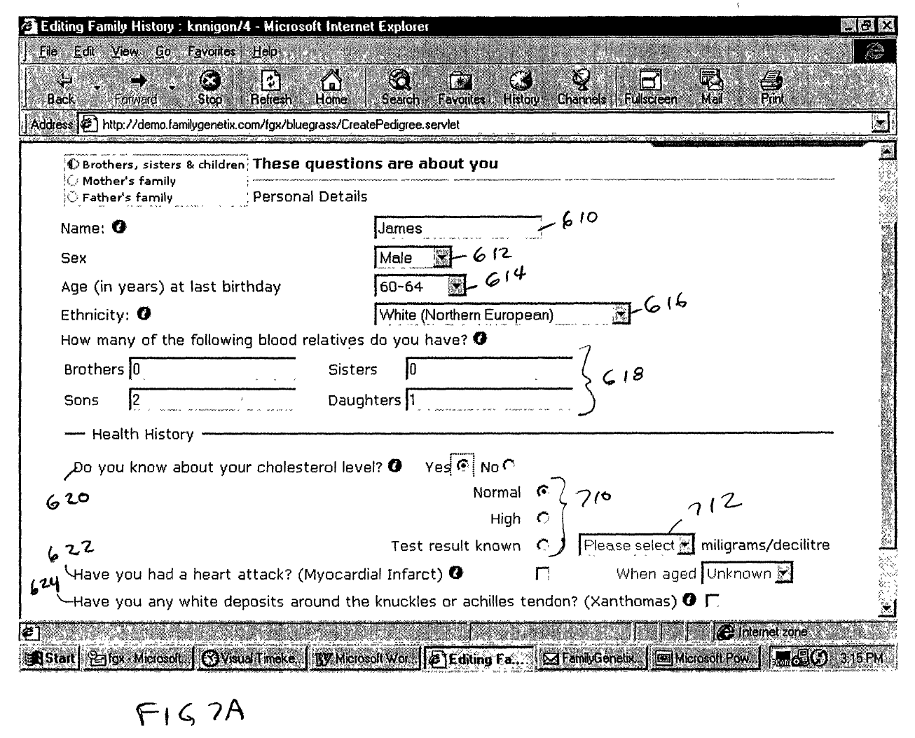

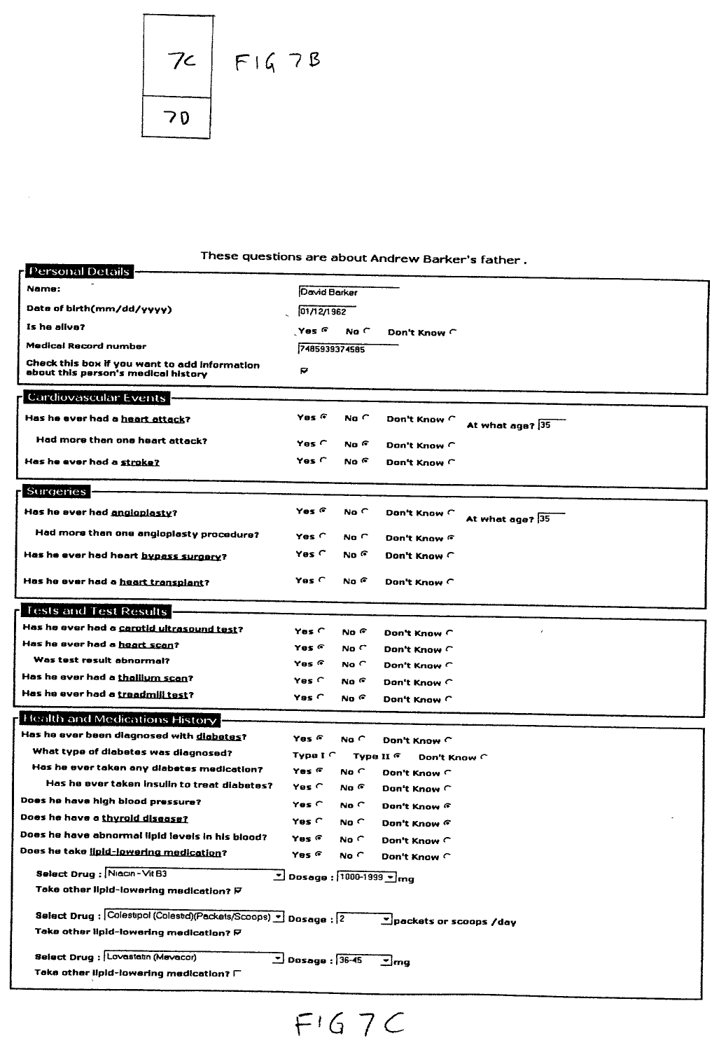

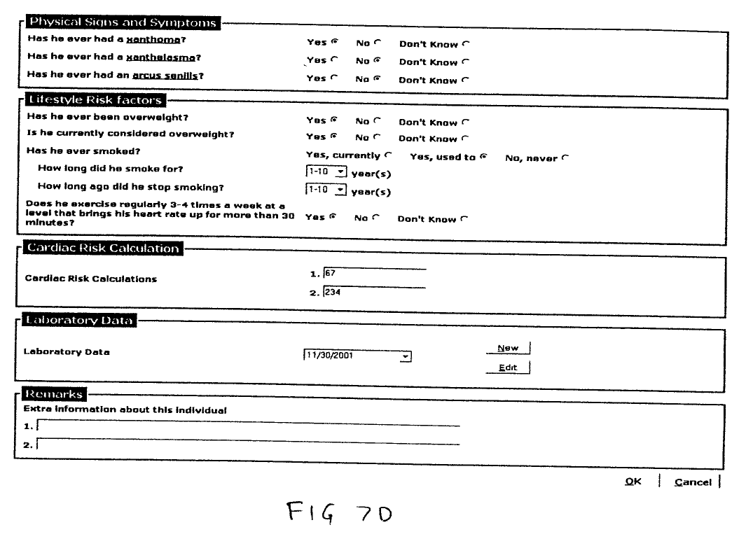

The screen changes dynamically when data is entered. For example, FIG. 7A shows a partially filled-out form, such as that shown in FIG. 6A, for the root individual. Note that the user has selected the radio button indicating “yes” for question 620, indicating that his/her cholesterol level is known. In response to this selection, the form changes to include radio buttons 710 which categorize the cholesterol level as “Normal,” “High” or being known from a test result. This last radio button also has a drop-down menu 712 that allows the user to select a cholesterol range from a menu of possible ranges. FIGS. 7B through 7D show details from the form shown in FIG. 6B. The form shown in FIGS. 7B through 7D illustrates how more complex medication data may be collected and managed by clinical users or other clinical systems. In addition, the form shown in these Figures illustrates the expansion of questions to allow the user to gather more detailed information. For example, on smoking habits, on the basis of earlier answers to questions shown in the form of FIG. 6B.

Referring once again to FIG. 4, after obtaining the personal details, heart attack history, cholesterol level and physical signs indicative of FH from the root individual at step 410, the process next executes step 412 which determines if the root individual has entered numbers in the children fields 618. If so, step 414 is executed which cycles through screens similar to those shown in FIGS. 6A through 7D to collect personal details, heart attack history, cholesterol level and physical signs indicative of FH for each child.

After the information on the root individual and his/her children has been entered, the process, at step 416 displays the partial family tree and asks the user if any information needs to be edited. This process is described below with reference to FIGS. 4A, 5, 8A, 8B and 9. After allowing the user to edit the information about the root and the root's children, the process determines, at step 418 if the root has siblings. If so, then, at step 420, the process collects the same information about each sibling and each child of each sibling. After step 420, or after step 418 if the root has no siblings, step 421 displays the current family tree and asks the user to review and edit it.

Next, at step 422, the process asks for information about the root's mother and father. A form such as that shown in FIGS. 6A through 7D is completed for each parent. Next, the process determines, at step 424, if the mother has any siblings. If she does, then, at step 426 the process collects information about each of the siblings. After step 426 or, if the root's mother did not have any siblings, after step 424, the process executes step 428 which again displays the partial family tree and asks the user to review and edit the information.

Steps 430, 432 and 434 perform the same functions as steps 424, 426 and 428 except for the root's father. Next, at step 436, the process obtains information about the father's parents and the mother's parents. At step 438, the process displays the family tree and asks the user to edit it for the last time in the data entry process. After the user has reviewed and edited the family history, the data entry process ends at step 440. While the display and edit function is shown as being invoked at several points in the process, it is contemplated that it may be invoked fewer times, for example, only after all of the family history information has been entered, or more times, for example, after information has been entered about each individual.

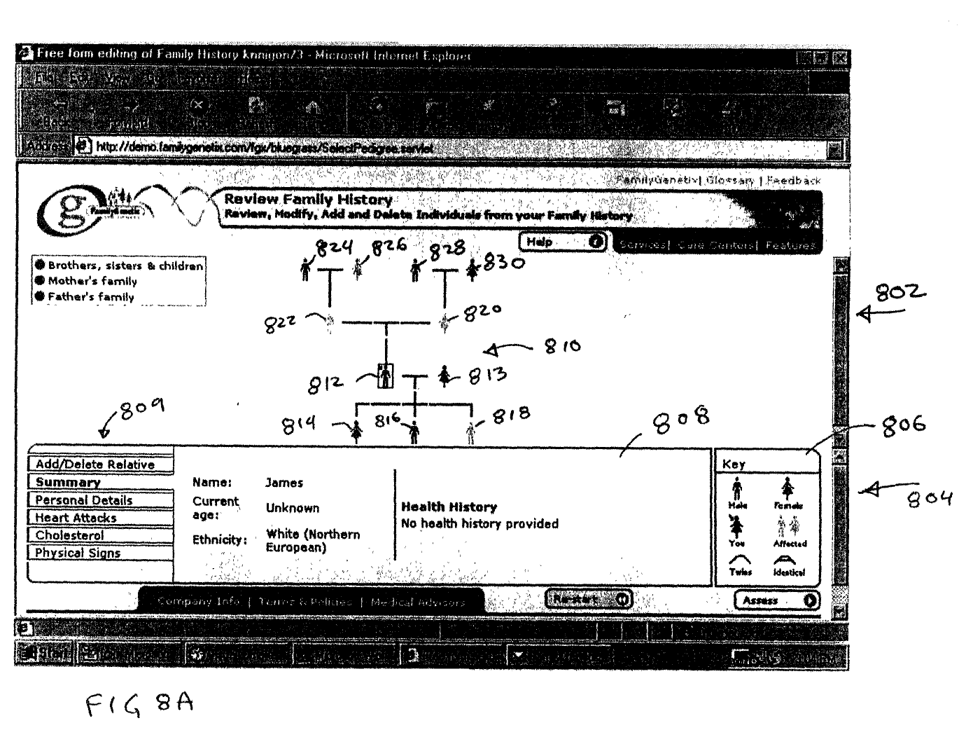

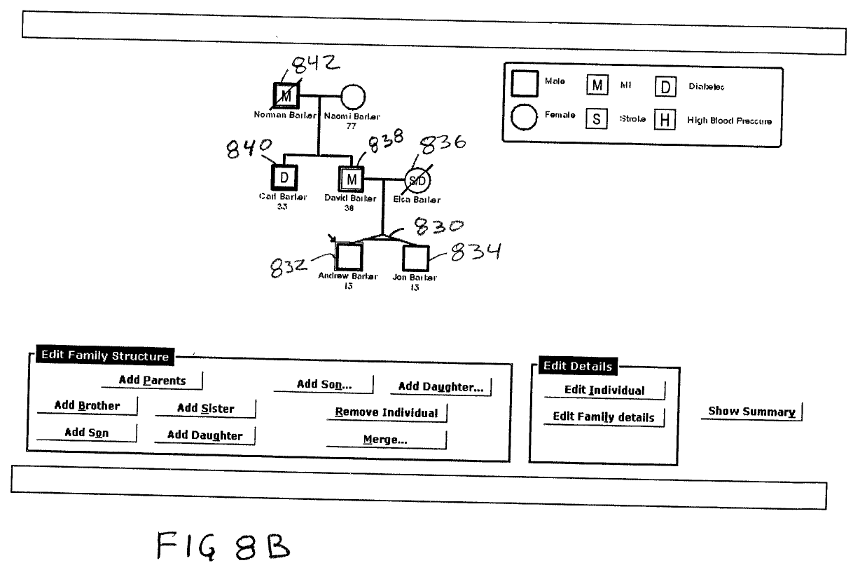

FIG. 4A is a flow-chart diagram that describes the display and edit function performed at steps 416, 421, 428, 434 and 438. An exemplary screen print of produced by the display and edit function is shown in FIG. 8A. At step 442, the function displays the family history 810 in an upper frame 802 of the web page shown in FIG. 8A and displays an edit box 808 in a lower frame 804. The exemplary family history highlights the root individual, James, and the edit box displays his personal details. The family history 810 is in the form of a family tree with a male or female icon for each member of the family. In addition to James, 812, The exemplary family history includes his three children 814, 816 and 818; his wife 813, his parents 820 and 822 and his grandparents 824, 826, 828 and 830. Lower frame includes a key 806 that aids in interpreting the family history 810. As indicated by the key, affected individuals, in this example individuals who have FH are indicated in a different color than non-affected individuals. From this display, it can be seen that both of James's parents, one of his grandparents and one of his sons have high cholesterol.

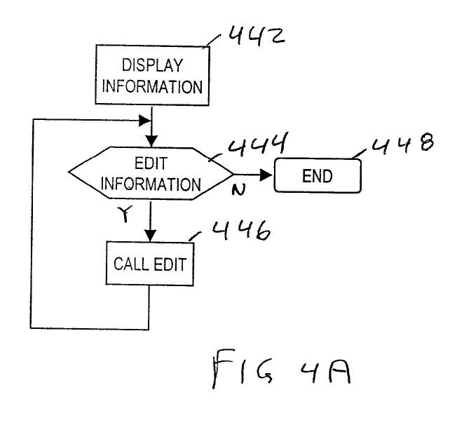

Returning to FIG. 4A, once the information is displayed, the user, at step 444, can choose to edit the family history data. If the user chooses to edit the information, he/she selects an individual from the family tree, for example, by double-clicking a pointing device such as a computer mouse, while the pointing device indicates the individual. Once the individual is selected, the edit process, described below with reference to FIG. 5, allows the user to change the personal details as well as the illness-specific information for that individual. After editing the information for the individual at step 446, the function branches to step 444 to either select another individual for editing or to indicate that no more editing is needed and end the function at step 448.

FIG. 8B illustrates a family history or pedigree as it may be viewed by an expert (e.g. a clinician). This display uses standard symbols. For example, a twinship symbol 830 is shown between the symbol 832 for Andrew Barker and the symbol 834 for Jon Barker. In addition, the symbol 836 for Elsa Barker indicates that she had both diabetes and a stroke and is now deceased. The symbol 838 for David Barker shows that he had a myocardial infarction (MI) while the symbol 840 for Carl Barker indicates that he has diabetes. The symbol 842 for Norman Barker shows that he had MI and is now dead. While the symbols used in FIG. 8B use letters to designate the various conditions, it is contemplated that colors may be used instead. For example, MI may be indicated as chartreuse, stroke may be indicated as pea-green, diabetes may be indicated as purple and high blood pressure may be indicated as blue.

Although not shown, the system may also represent childbearing incestuous and other more distant consanguineous relationships, for example where a son is the father, through his mother of a half sibling.

The Family History Editor is one aspect of the inventive system. This tool allows the user to review, modify, add and delete individuals. This means that the user can: check the information held on each person is correct; change the information on individuals if it is incorrectly entered or incomplete; add a relative not shown on the family history diagram; or delete a person who should not be shown. First-time users of the system often provide incomplete data simply because they do not anticipate the particular questions that will be asked. The family history editor allows this user to enter the partial data, research the missing data and then add the missing data to the partial data at a later time, without having to reenter the original data.

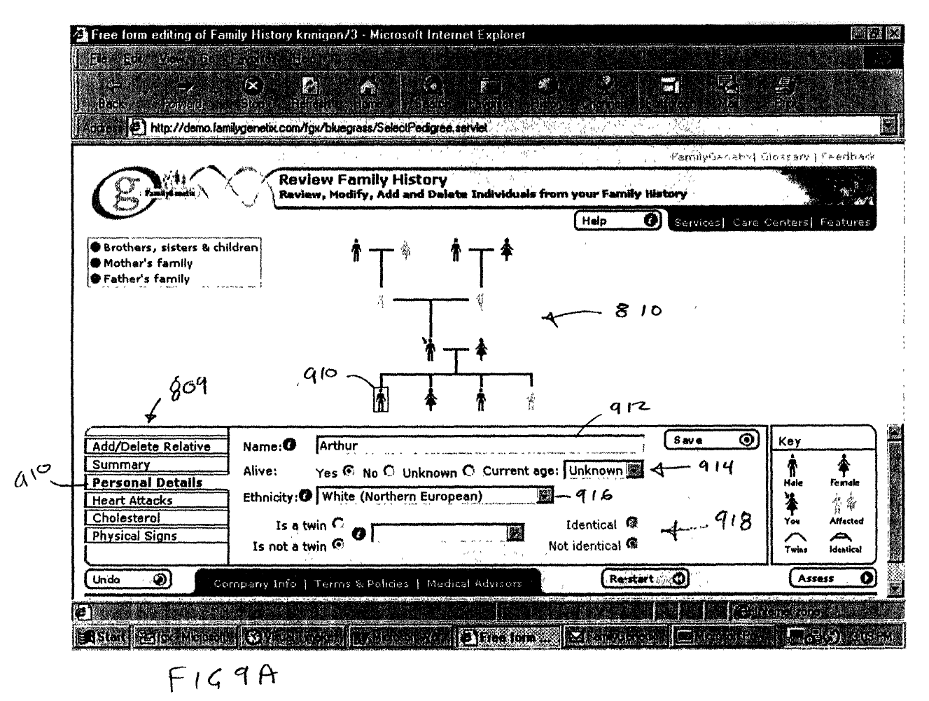

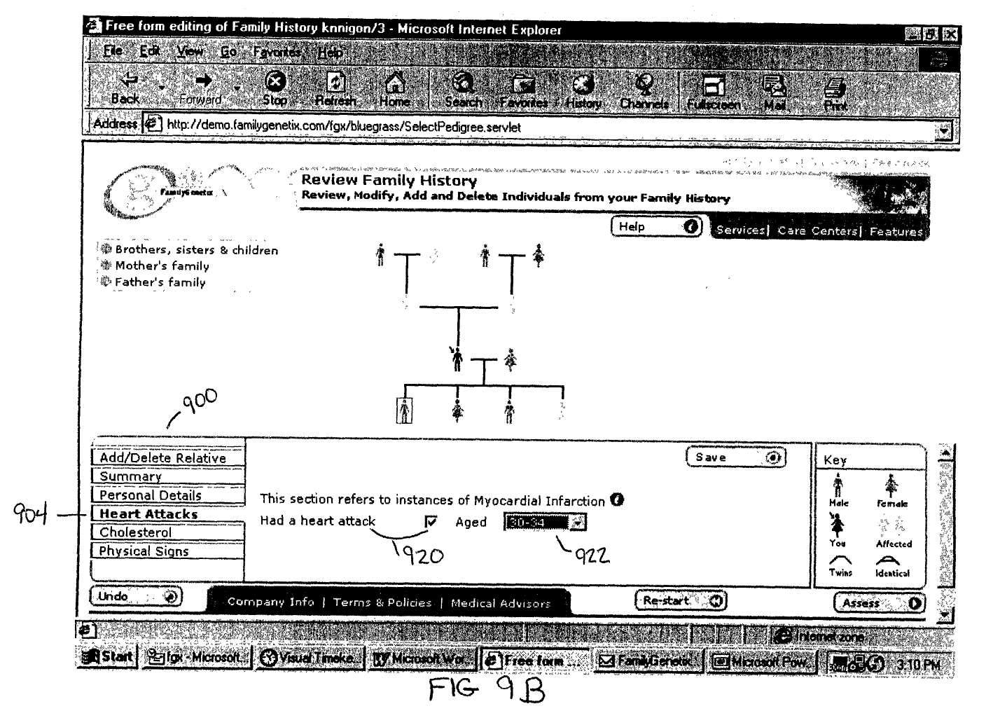

FIG. 5 is a flow-chart diagram of an exemplary family history editing process. With reference to FIGS. 5 and 8A, the process begins at step 510 by prompting the user to select one of the individuals displayed in the family history 810. When an individual is selected at step 512, the process displays the personal details of the individual in the edit box 808. At step 515, the user selects information to be edited from the menu 809 on the left side of the edit box 808. Using these menu entries for the FH example shown in FIG. 8A, the user can: 1) add or delete an individual from the family tree; 2) edit the individual's personal details; 3) modify the person's heart attack record; 4) change the information on the individual's cholesterol level; and 5) change the information on the individual's physical signs of FH. In the example shown in FIG. 9A, the user elects to add another son, Arthur. The edit process adds an icon for Arthur to the family tree and then prompts the user to enter Arthur's personal details 910. The user enters Arthur's name into the field 912, enters either his current age or his status as deceased in field 914, enters his ethnicity in field 916 and enters his status as a twin or not a twin in field 918. In FIG. 9B, the user then elects to enter Arthur's heart attack information. This causes the system to display a check box 920 which the user selects to indicate that Arthur has had a heart attack and a drop-down menu 922 from which the user selects an age range in which the heart attack occurred.

Once the selected information has been edited at step 516, it is stored, at step 518, into the family history database 138 (shown in FIG. 1). At step 520, the user either continues to edit the information by branching to step 510 to select another individual or terminates the process at step 522.

The process described above with reference to FIGS. 2 through 9B continues until all of the family history information known to the user has been entered. Having entered information on the prevalence of the disease in their family, the user can proceed to a full report on their disease of interest.

The processes and systems described thus far configure and present questions and configure and store resulting data. A third component of the system is the manipulation of the stored data to “create medical knowledge.” Analysis is supported in the exemplar system of this invention as “plug-ins” or separate modular computer code entities that can act within an “analysis server” (part of the present invention). This can happen at a number of levels.

Data items may be summarized (and may be stored) for subsequent presentation. For example, a rolling average of blood pressure at diastole may be calculated and stored by a simple analysis “plug-in.”

Statistical risk analysis. In a number of key diseases, such as Breast Cancer and Ovarian cancer, public domain models have been created, based on large population studies, also in the public domain, that allow, based on family disease histories, such as those collected by the present invention, the age dependent risk of an individual developing a disease, the likelihood that they carry a certain gene etc. to be calculated. The present invention makes use of these models as plug-ins to its analysis server.

Heuristic risk analysis. Various bodies of medical opinion have created public domain guidelines for the identification of those at risk of developing diseases. Medical experts may encode these guidelines as a set of computer processable rules. The present invention allows these rules to be included either as separate, disease specific analysis “plug-ins” or within a generic rules processing engine (not part of the present invention) which may, itself, be treated as an analysis “plug-in” to the present invention.

Combined Statistical Heuristic. The inventors have determined that the capability of the present invention may include the output of a statistical risk assessment as one of the factors within a heuristic risk assessment in genetic disease. This has, for example, been important in encoding the Oxford guidelines for breast cancer screening which make use of a statistical variable within a heuristic in determining the screening needs of women at risk of breast cancer.

The report, provided by the system to the user, contains content selected on the basis of the risk analysis or analyses performed by the system. The report is specific to the family history and pattern of disease reported by the user. The report delivers an over-all summary of risk, supplemented by more detailed guideline-driven and statistical explanations of risk. The user is provided with advice on how to improve their outlook, and on the next steps they may wish to consider:

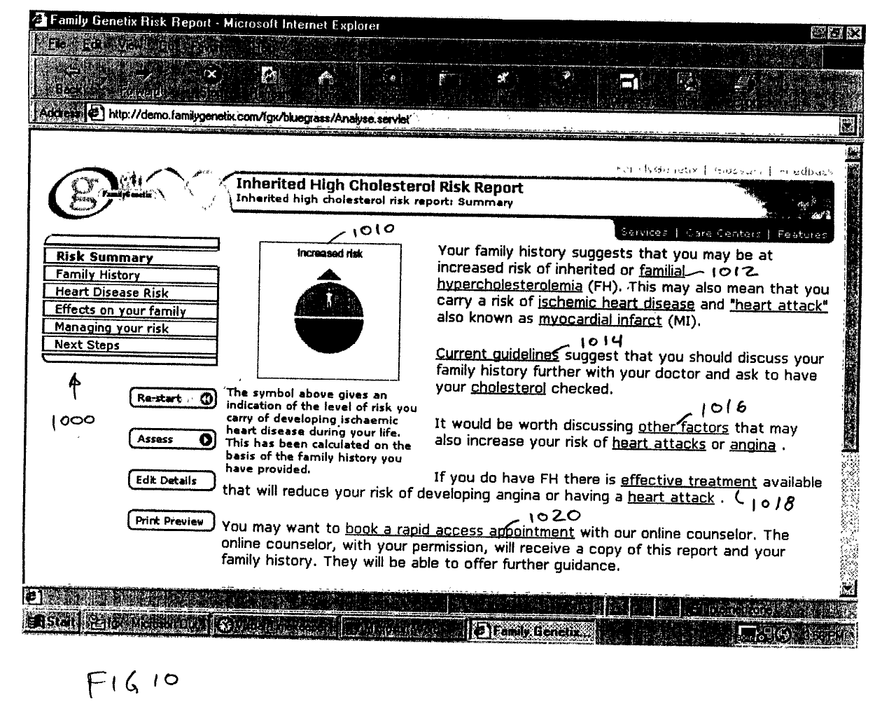

FIG. 10 is a screen print of an exemplary report for James in the example given above. The report includes an indicator 1010 that points up to indicate that James's family history indicates an increased risk of his contracting FH. The invention reported herein can make use of a variety of mechanisms for visual communication of risk of which this is one. In addition to the indicator 1010, the report includes explanatory text that includes hyperlinks through which the user may find out various information relevant to the disease. This includes: more information on familial hypercholesterolemia 1012; information 1014 on current guidelines for actions to take to monitor cholesterol; information 1016 on factors other than heredity that may affect James's chances of contracting FH and information 1018 on treatment options for FH.

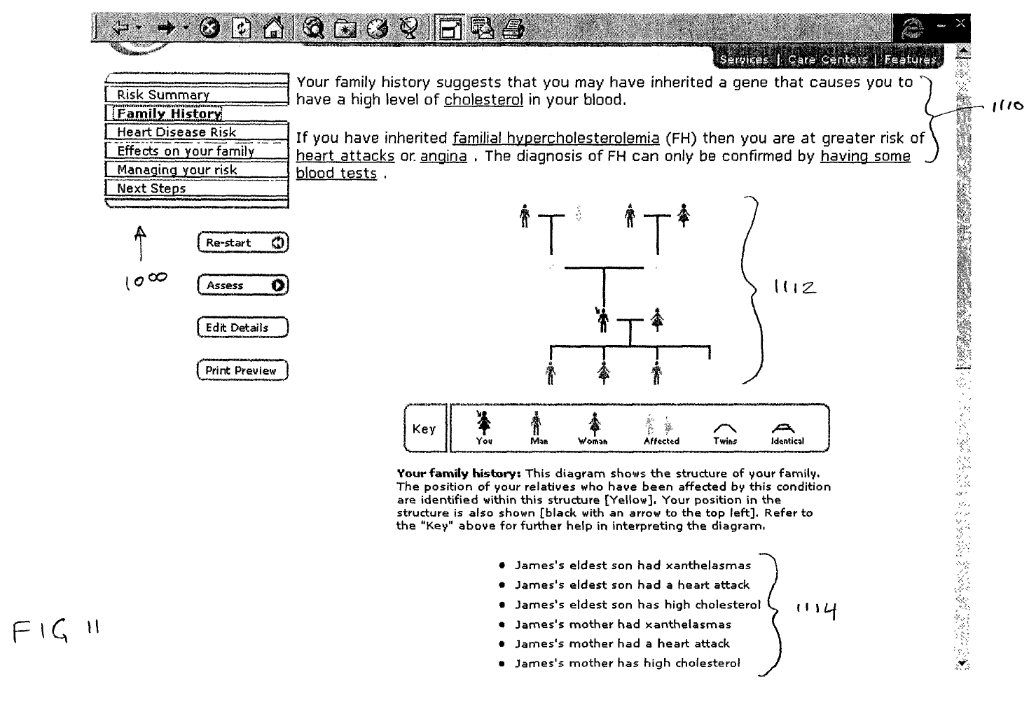

If, from the display shown in FIG. 10, the user selects “family history” from the menu 1000, a display such as that shown in FIG. 11 is produced. This display includes a text summary 1110 of the report shown in FIG. 10, a depiction of the user's family tree 1112 and specific items 1114 selected from the family tree that support the analysis presented in the report shown in FIG. 10. This report may be specially effective as it personalizes the risk to the individual, showing how the risk is derived from the individual's relatives.

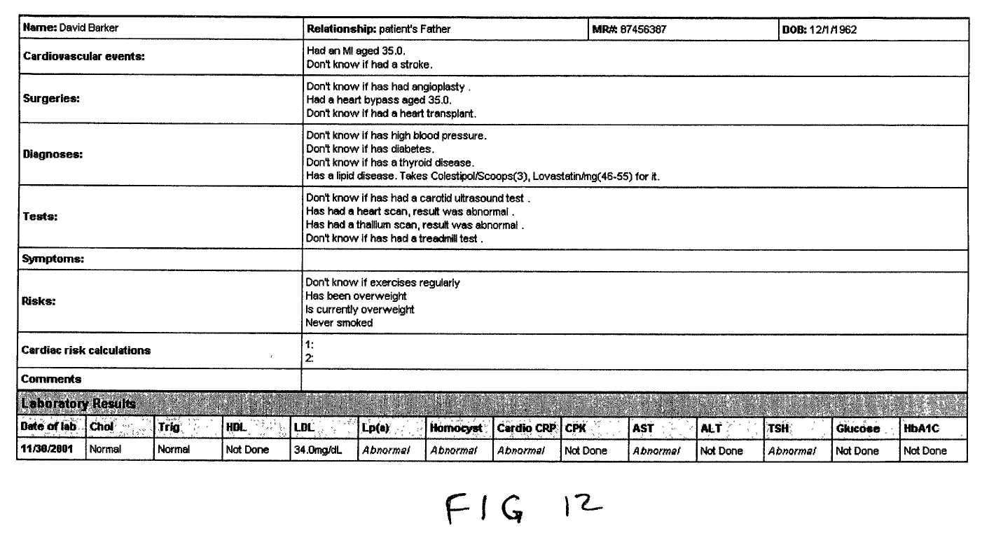

FIG. 12 is an extract from a more detailed report that may, for example, be presented to a clinician. This report has a format that has been specified by the particular client using a report generator. This report shows additional detail concerning an individual from a pedigree that may be viewed by a clinician or other expert.

In addition to this information, the report web page, shown in FIG. 10, allows the user to schedule an appointment with a genetic counselor. As set forth above, the counseling service enables a user to obtain counseling in many forms: online counseling either by an exchange of emails or a telephone consultation with a board certified counselor, or a referral into a trained counselor for a personal appointment booked online through the inventive service.

The counselor will, with the permission of the user, obtain a family history collected by the Risk Assessment Service. This offers a huge time saving compared with traditional genetic counseling, where at least half the consultation time is spent in gathering the information needed to carry out the consultation. In a consultation, the user and the counselor can analyze and extend the family history record. The family history record is then available for subsequent work and can be integrated with other healthcare provider systems, or made available, with permission, to other healthcare professionals. The counseling service may be provided by a combination of professionals employed by the healthcare organization and partner organizations.

The exemplary genetic information service is a content-rich source of information on the latest developments in genetics, explaining in a clear and accessible way and in many languages, the implications of the latest research. The content has been commissioned from leading authorities in the field, but reviewed for clarity by experts in the use of “plain language” in educating the public about healthcare issues. Geneticists, counselors and researchers desirably are able to draw family histories as a way of organizing and managing the family history information of a patient. The family history information entered by the user is the first step in carrying out an analysis because: 1) It provides a simple representation of all critical medical information, 2) it enables a user to see at a glance all critical and relevant biological links, and 3) it enables a user to make assessments of the further evaluation or testing that may be desirable.

The present invention includes a set of facilities that enable a user to organize all of the information associated with a family history and, in addition, carries out a comprehensive genetic risk assessment using “plug-in” applications.

Much value is added here—existing plug-ins for statistical risk assessment were developed for use by expert users and do not present an easy to use interface such as that described here. The ease of use of the inventive system is one of its key advantages over using only this complex risk assessment software.

Although the exemplary embodiment of the subject invention has penetrance data sets only for breast cancer and ovarian cancer penetrance for other diseases may be generated from population data, thus it is contemplated that penetrance data may be available for all genetically-linked diseases handled by the system.

Segregation analysis aims to define the most probable genetic mechanism (if any) involved in causing a disease. This involves comparing the observed patterns of the disease in a collection of well-verified family histories known scientifically as “pedigrees.” These family histories are collected from families in which the disease of interest has been known to occur. It has been used for several purposes including: gene mapping calculations, genetic counseling, segregation analysis and paternity testing.

There are a number of models for genetic risk ranging from the simple patterns discovered by Gregor Mendel at the beginning of the 20^{th} century, to the more complicated polygenic patterns studied in the 1980s and 1990s. The aim of segregation analysis is to determine which of these models fits best. Once a model has been tested and shown to be correct by maximum likelihood comparisons, it can be used to provide a method for estimating the cancer risk for individuals in a family in clinical practice.

For all but the simplest pedigree structures, risk calculations are complex and are, therefore, best encapsulated in computer programs. As described below, using the guideline authoring capability, the inventive system can make use of the preferred clinical guideline and data set of the purchasing organization.

The exemplary embodiment of the invention described herein, makes use of data from the Houlston data set for segregation analysis, described below, and of the diagnostic guidelines from the American College of Medical Genetics and the New York State Department of Health. The risk of breast cancer has been investigated in a large population-based, case control study conducted by the Centers for Disease Control, known as the CASH (Cancer and Steroid Hormone) study. The data set was based on 4,730 cases of breast cancer in the age range 20-54 confirmed by pathologists. The control group consisted of 4,688 matched on geographic region and age category. Family histories were obtained from the study and control groups concerning breast cancer history in the family. The exemplary embodiment of the invention uses the Houlston method to estimate probable risks associated with inherited breast cancer.

For ovarian cancer, the exemplary embodiment of the invention makes use of data generated by D. Eccles and colleagues. A consensus panel brought together by the NIH produced the clinical guideline used in ovarian cancer.

A total of 314 family histories were analyzed, containing 1020 “nuclear families.” 875 cancers were recorded, of which 346 were ovarian cancers. Liability classes were calculated. Eccles and colleagues have, subsequently, produced a data set suitable for use in segregation analysis. The NIH consensus panel suggested that, for those with a 5% or less lifetime risk, there was no definite evidence for screening but that they may benefit from referral into clinical trial. This would be typical of families with a single close degree relative who developed ovarian cancer at any age.

For those with a lifetime risk of 7% or more or with a 3% risk of hereditary cancer syndrome, referral for further investigation is required. This would be typical of families where 2 or more family members have had ovarian cancer. The inventive service encourages users to be aware of the risks that they face as a result of their family history and therefore encourages them to change their habits in order to improve the quality of their lives. Because the analysis is based on very personal data entered by the user, it is contemplated that the recommendations provided by the inventive system will have more weight than typical statistical arguments.

For colon cancer, the exemplary embodiment of the invention makes use of the guideline from the American Cancer Society Colorectal Task Force (Anderson and colleagues, Wisconsin Medical Journal). These guidelines associate risk with: 1) colorectal cancer or adenomatous polyps in 1 1st degree relative<60 years and 2) Colorectal cancer in 2 or more 1st degree relatives.

For familial hypercholesterolemia, the exemplary embodiment of the invention makes use of the guidelines from the World Health Organization Human Genetics Program. This associates risk with: 1) total cholesterol greater than 260 mg/dl if under age 16, 2) total cholesterol greater than 290 mg/dl if over age 16, 3) LDL-cholesterol greater than 190 mg/dl if over age 16 4) Xanthomata (white cholesterol deposits on skin over peripheral joints) in first or second degree relatives, 5) Family history of Myocardial Infarct (MI) under age 60 in a first degree relative, 6) Family history of Myocardial Infarct (MI) under age 50 in a second degree relative, or 7) Family history of total cholesterol greater than 290 mg/dl in a first or second degree relative

In addition to breast cancer, ovarian cancer, colon cancer and familial hypercholesterolemia, the subject invention data sets may added to the invention to screen for other genetically linked diseases such as Type 2 Diabetes, osteoporosis, asthma, obesity, sickle cell anemia, Alzheimer's disease, hypertension, attention deficit disorder, testicular cancer and birth defects.

The present invention can provide heuristic risk assessment, and also allows new clinical guidelines to be developed for the genetic risk assessment software. Again, the present invention is not dependent on but can make use of a variety of clinical guideline authoring tools or similar technologies as methods for supporting heuristic risk assessment.

Genetic counseling is the key discipline in medicine that is based on historical (family history and lifestyle) information, rather than immediate symptoms and observations. Genetics is, through accumulating knowledge, becoming more important in other medical disciplines, e.g. oncology, cardiology, neurology etc. Genetic counseling as currently delivered, however, is inefficient due, in the main, to the methods used to collect family histories. Half of the consultation time can be taken up by obtaining data for, drawing and reviewing the family pedigree on which the assessment of the patient's risk is made.

The present invention increases the quality and scope of genetic counseling while reducing its cost 1) by enabling more consultations to take place with trained professionals, rather than other healthcare workers who have no formal training in genetics; 2) by providing counselors who have developed a specialty in dealing with those genetic diseases that manifest themselves in adulthood as opposed to the traditional pre-natal field; and 3) by using the internet and telephone to deliver counseling for around a tenth of the cost of the traditional service.

The inventive service is also extremely convenient to the patient as it can be offered where and when the patient desires to use it, from the privacy of his or her own home or within a referral center. The inventive Risk Assessment Service has targeted those conditions for which it is possible to reduce the cost of chronic care through prevention, earlier diagnosis and treatment.

The exemplary embodiment of the inventive Risk Assessment Service has targeted those conditions for which risk can be reduced by preventative treatment and lifestyle changes. The system also provides an effective vehicle for encouraging patients with a high-risk assessment to lower their risk by pursuing such treatment and/or lifestyle changes. This allows managed care organizations (MCO's) to secure better outcomes for their members and at the same time reduce the cost of chronic care by a relatively small increase in preventative care. Thus, the MCO's will be able to make overall savings by introducing such a program.

The present invention enables healthcare providers to: 1) shape the genetic content that their members access and thus better manage member expectations of genetic services and 2) enhance the image and reputation of the plan as a sponsor of preventative medicine and early detection. Surveys show that members want telemedicine and are prepared to move to those providers who are able to offer it. Thus, the present invention is at the forefront of the telemedicine revolution. MCO's that implement service in accordance with the subject invention are able to attract customers who defect from less techno-friendly health plans, and retain their existing customers.

By presenting a family history documenting and analysis system that is secure and easy to use and that also provides the user with easy to read peer reviewed literature on several genetically linked diseases, the subject invention promotes prevention by helping to identify high risk individuals at an early stage.

While the invention has been described in terms of exemplary embodiments, it is contemplated that it maybe practiced as outlined above with modifications within the scope of the following claims.

What is claimed:

1. A computer aided method for determining whether an individual is a susceptible to contracting a genetically-linked illness comprising the steps of:
establishing a connection between the individual in a first computing environment and a data gathering and evaluation system in a second computing environment, distinct from the first computing environment;
receiving, at the second computing environment, a selection from the individual of a particular genetically linked illness from a plurality of genetically linked illnesses;
retrieving data relevant to the specific illness from a first database accessible to the second computing environment;
prompting the individual for family history data specific to the selected illness, receiving the family history data provided by the individual and storing the received family history data into a second database accessible to the second computing environment;
analyzing, in the second computing environment, the received family history data using the retrieved data relevant to the selected illness to determine the susceptibility of the individual to the selected illness; and
generating a report including an indication of the susceptibility of the individual and transmitting the report to the first computing environment.

2. A method according to claim 1, further comprising the step of prompting the individual to receive genetic counseling if the report indicates that the individual is susceptible to the selected illness.

3. A method according to claim 1, wherein the first database includes educational materials relevant to the selected illness and the method further includes the step of presenting the educational materials relevant to the selected illness to the individual for review at the first computing environment.

4. A method according to claim 1, wherein the first computing environment is remote from the second computing environment and the step of establishing the connection between the first computing environment and the second computing environment further includes the step of establishing a secure connection between the first computing environment and the second computing environment.

5. A method according to claim 1, further including the step of storing any information that identifies the individual only on the first computing environment and associating a unique identifier with the individual and the family history data stored in the second database.

6. A method according to claim 1 wherein the step of generating the report includes the step of including specific data from the family history in the report.

7. A method according to claim 1, wherein the step of analyzing the received family history data includes the step of summarizing the family history data to generate summary data and storing the summary data.

8. A method according to claim 1, wherein the step of analyzing the received family history data includes the step of performing a statistical risk analysis process on the family history data.

9. A method according to claim 1, wherein the step of analyzing the received family history data includes the step of performing a heuristic risk analysis process on the family history data.

10. A method according to claim 9, wherein the step of analyzing the received family history data further includes the step of performing a statistical risk analysis process on the family history data.

11. A computer aided method for determining whether an individual is a susceptible to contracting a genetically-linked illness comprising the steps of:
establishing a connection between the individual in a first computing environment and a data gathering and evaluation system in a second computing environment, distinct from the first computing environment;
receiving, at the second computing environment, a selection from the individual of a particular family history;
retrieving and displaying data relevant to the specific family history and retrieving information relevant to a genetic condition associated with the family history from a database accessible to the second computing environment;
receiving information from the individual which modifies the specific family history;
analyzing, in the second computing environment, the modified family history data to determine the susceptibility of the individual to the genetic condition; and
generating a report including an indication of the susceptibility of the individual to the genetic condition and transmitting the report to the first computing environment.

12. A scalable computer architecture for an on-line genetic testing system comprising:
a presentation server which establishes respective connections with a plurality of users and includes a further plurality of input forms for obtaining family history information and a plurality of output forms for reporting results of family history evaluation;
an applications server which includes:
means for receiving data entered by the plurality of users;
means for retrieving data relevant to each user from at least one database;
means for selecting from among the plurality of forms, a respective plurality of the input forms to be sent to the plurality of users in order to obtain family history data received from the plurality of users;
means for storing the family history data into the at least one database;
means for analyzing the family history data to determine a risk for genetic illness; and
means for selecting from among the plurality of forms, a respective plurality of the output forms and for entering data from the analysis of the family history into the output forms; and
a database server which holds the at least one database.

13. A scalable computer architecture according to claim 12, further including:
means for eliciting family history data collection in an accessible, convenient and appropriate manner from at least one of: a) members of the public, b) members of the medical professions who are not expert geneticists and c) expert geneticist clinicians and researchers.

14. A scalable computer architecture according to claim 12, further including:
means for presenting risk analysis and other family history based data an accessible, convenient and appropriate manner to at least one of 1) members of the public, 2) members of the medical professions who are not expert geneticists and 3) expert geneticist clinicians and researchers.

15. A scalable computer architecture according to claim 12, further comprising an access control mechanism which implements a permission scheme to allow appropriate access to the stored family history data to the patient who entered the data, clinical genetics specialists, non-genetics-expert clinicians and systems management personnel and to control access to the family history data by each of these individuals.

16. A scalable computer architecture according to claim 12, wherein the family history data includes a plurality of family histories.

17. A scalable computer architecture according to claim 12, wherein the presentation server, the applications server and the database server are implemented on a single computer system.

18. A scalable computer architecture according to claim 12, wherein the presentation server, the applications server and the database server are implemented on respectively different computer systems.

19. A scalable computer architecture according to claim 18, wherein the applications server includes a control server and an analysis server which are implemented on at respectively different computer systems.

20. A scalable computer architecture according to claim 18, wherein each of the presentation server and the applications server are implemented using multiple computer systems.

21. A method for regulating the allocation of genetic testing resources among members of an organization, the method comprising the steps of:
performing on-line family history evaluation to identify individuals from the organization who may be susceptible to genetically related illnesses;
automatically scheduling genetic counseling for each of the identified individuals to determine if the individual is a candidate for genetic testing; and
scheduling genetic testing for any individual determined to be a candidate during the genetic counseling.

22. A method according to claim 21, wherein:
the step of performing the on-line family history evaluation includes the steps of:
prompting a user to enter family history information concerning one of the members of the organization, the information including data related to a specific genetic illness;
developing a family tree for the one member, including risk factors for each entry in the family tree; and
analyzing the family tree to determine a risk factor for the one member and reporting the risk factor to the user; and
the step of automatically scheduling genetic counseling for each of the identified individuals includes the step of transferring the respective family tree for each of the identified individuals to a respective one of the genetic counselors.

23. A method according to claim 21, further including the step of providing each of the identified individuals with educational material on the genetically related illnesses before automatically scheduling the genetic counseling.

24. A method according to claim 21, wherein the step of automatically scheduling genetic counseling includes the step of prompting the user to select counseling from a group consisting of on-line counseling, telephone counseling and in-person counseling.

25. A method for regulating the allocation of genetic testing resources among members of an organization, the method comprising the steps of:
performing on-line family history evaluation to identify individuals from the organization who may be susceptible to genetically related illnesses;
scheduling genetic testing only for individual determined to be a susceptible to genetically related illnesses by the on-line family history evaluation.
